(19) United States
(12) Patent Application Publication
(10) Pub. No.: US 2010/0016172 A1
(43) Pub. Date: Jan. 21, 2010

(54) Nucleic acid binding assay materials and methods

(76) Inventor: Aseem Z. Ansari, Madison, WI (US)

Correspondence Address: PIONEER PATENT, LLC, P. O. BOX 128, NORTHBROOK, IL, 60065-0128, US

(21) Appl. No.: 12/009,078

(22) Filed: Jan. 16, 2008

Related U.S. Application Data
(60) Provisional application No. 60/880,933, filed on Jan. 16, 2007.

(51) Int. Cl. C40B 30/04; C07H 21/04; C40B 40/08

(52) U.S. Cl. 506/9; 536/23.1; 506/17

(57) ABSTRACT

The invention provides reliable, reusable materials, system and methods for use in nucleic acid binding assays between a nucleic-acid binding assay molecule and a robust unimolecular target. In a preferred embodiment, the target is a nucleic acid-containing molecule, L¹-X¹-L²-X² (where L¹ is the linker to the solid support, and L² is the linker between the two nucleotide-pairing regions, X¹ and X²; L² folds such that double-stranded DNA structure forms between X¹ and X²). L² is a polynucleotide three nucleotides in length, having the sequence GNA, where N is A, G, C, T, or U. A typical assay uses a target array, where multiple targets are bound to a solid surface. The linker, L¹, of the nucleic acid target to the supporting surface is useful although it is significantly longer than suggested in the art. The length of L¹ is preferably 8 to 30 nucleotides in length, more preferably from 10 to 20 nucleotides.

### CROSS-REFERENCE TO RELATED APPLICATIONS

The present patent application claims the benefit of U.S. Provisional Patent Application No. 60/880,933, filed Jan. 16, 2007.

### DESCRIPTION

Methods, materials and systems are disclosed for the assay of nucleic acid binding of molecules. Nucleic acid binding molecules assayed include DNA-binding proteins and molecular mimics. An array made up of target nucleic acid with known sequence variations is treated with the molecule to be assayed for nucleic acid binding activity. Analysis of the binding to the array includes study of the bound sequences and intensity of binding to those sequences to identify the nucleic acid binding sites.

### BACKGROUND OF THE INVENTION

The present invention is related to improved methods and materials for the assay of nucleic acid binding of molecules. Molecules, including proteins, which act on nucleic acids have biological functions that, in many cases, are critical. For example, with respect to the genome, nucleic acid binding molecules play a critical role in accessing, deciphering and expressing genomic information (RNA and DNA). Chromosomal stability and maintenance is also under the purview of DNA-binding proteins. RNA binding by molecules is also an important area of work. The tools available in the art have led to limited success. Beyond the ability to describe the structural motifs of several known DNA-binding proteins and their modes of DNA binding, attempts to define general rules of DNA recognition have met with little success. There are a great many as-yet-unidentified molecules that play key nucleic acid-binding roles in biological processes. There is a need for new materials, systems and methods for identifying new nucleic acid-binding molecules.

There is a need for new materials, systems and methods for ascertaining the detailed effects of sequence variation on nucleic acid binding by a nucleic acid-binding molecule. Some of the sequences bound are regulatory elements. There is a need for a route to reaching a key goal of modern biology—the identification of regulatory elements in genomes. To further understand the biological role of nucleic acid-binding molecules, it is vital to determine their sites of action in the genome.

A related central goal of synthetic biology, chemical biology, and molecular medicine is the design and creation of synthetic molecules that can target specific DNA sites in the genome. Such molecules are useful in vitro and in medicine to regulate biological processes such as transcription, recombination, and DNA repair. A major hurdle in the design of new classes of DNA binding molecules is the inability to comprehensively define the full range of their DNA sequence recognition properties and therefore to predict their potential target sites in the genome.

Given the importance of understanding the basis of molecular recognition between DNA and its nucleic acid binding molecules, several methods have been developed to determine the sequence specificity of DNA-binding proteins or drugs. The most frequently used is the SELEX approach, which utilizes selection and enrichment of the DNA sequences that bind with the highest affinity to a molecule of interest (Tuerk, C. & Gold, L. (1990) Science 249, 505-510). This assay, though highly informative, identifies only the best binding sequences while the less optimal, and often biologically relevant, sequences are missed.

Other commonly used biochemical or biophysical approaches to determine sequence specificity of nucleic acid-binding molecules are labor intensive and can only be used to study a limited set of sequence variants (Tuerk, C. & Gold, L. (1990) Science 249, 505-510; Fried, M. & Crothers, D. M. (1981) Nucleic Acids Res. 9, 6505-6525; Garner, M. & Revzin, A. (1981) Nucleic Acids Res. 9, 3047-3060; Galas, D. J. & Schmitz, A. (1978) Nucleic Acids Res. 5, 3157-3170; Heyduk, T., Ma, Y., Tang, H. & Ebright, R. H. (1996) Methods Enzymol. 274, 492-503; Heyduk, T. & Heyduk, E. (2002) Nat. Biotechnol. 20, 171-176; Strauss, H. S., Boston, R. S., Record, M. T., Jr., & Burgess, R. R. (1981) Gene 13, 75-87). Medium-throughput microarrays have also been developed in which duplex DNA molecules are immobilized on surfaces with protein binding detected by surface plasmon resonance (Brockman, J. M., Frutos, A. G. & Corn, R. M. (1999) J. Am. Chem. Soc. 121, 8044-8051) or fluorescence (Bulyk, M. L., Huang, X. H., Choo, Y. & Church, G. M. (2001) Proc. Natl. Acad. Sci. USA 98, 7158-7163; Wang, J. K., Li, T. X. & Lu, Z. H. (2005) J. Biochem. Biophys. Methods 63, 100-110). Despite such demonstrations of feasibility, technical challenges have hindered the application of these array platforms. One of the most successful methods to date for determining sites of action of DNA-binding molecules is a solution phase, medium-throughput assay that utilizes DNA sequence variants presented in distinct wells and protein or small molecule binding, detected by displacement of a DNA-intercalating fluorescent dye (Boger, D. L., Fink, B. E., Brunette, S. R., Tse, W. C. & Hedrick, M. P. (2001) J. Am. Chem. Soc. 123, 5878-5891). Each of these medium-throughput approaches, however, is limited to querying DNA sequences with only 3-5 permuted positions. There is a need for technology with increased throughput materials and methods that are less materials- and labor-intensive. There is also a need for methods that permit increased sequence variation of possible nucleic acid binding sites.

More recently, chromatin immunoprecipitated (CHIP) DNA analyzed on oligonucleotide microarrays (chip) has been used to map binding sites for transcription factors in budding yeast Saccharomyces cerevisiae (Ren, B., Robert, F., Wyrick, J. J., Aparicio, O., Jennings, E. G., Simon, I., Zeitlinger, J., Schreiber, J., Hannett, N., Kanin, E., et al. (2000) Science 290, 2306-2309; Iyer, V., Horak, C. E., Scafe, C. S., Botstein, D., Snyder, M. & Brown, P. O. (2001); Nature 409, 533-538; Sikder, D. & Kodadek, T. (2005) Curr. Opin. Chem. Biol. 9, 3845). ChIP-chip is a valuable approach, yet it has several limitations including low signal-to-noise ratio, experimental variability, expense, labor-intensiveness, and antibody reactivity (Ren, B., Robert, F., Wyrick, J. J., Aparicio, O., Jennings, E. G., Simon, I., Zeitlinger, J., Schreiber, J., Hannett, N., Kanin, E., et al. (2000) Science 290, 2306-2309). Importantly, ChIP-chip studies have demonstrated that the in vitro affinity of transcription factors for specific DNA sequences is recapitulated in the occupancy of these sequences in vivo (Iyer, V., Horak, C. E., Scafe, C. S., Botstein, D., Snyder, M. & Brown, P. O. (2001) Nature 409, 533-538; Sikder, D. & Kodadek, T. (2005) Curr. Opin. Chem. Biol. 9, 3845). In the case of small molecules, the difficulty of ChIP-chip analysis is further compounded due to the lack of additional interpretive information such as comparative phylogenetic data. There is a need for materials and methods that would permit the determination of a full sequence recognition profile for a given molecule (e.g. small molecule, transcription factor, or a set of cooperatively binding factors) measured in vitro. Such data, in conjunction with computational approaches, would be highly instructive in computationally identifying binding sites in the genome. The present methods of the art, in the absence of genome-wide binding and expression data, limit the computational approaches to identifying regulatory sites to phylogenetic comparisons of conserved non-coding sequences (Kellis, M., Patterson, N., Endrizzi, M., Birren, B. & Lander, E. S. (2003) Nature 423, 241-254).

U.S. Pat. No. 5,556,752 (hereinafter the '752 patent) discloses DNA binding methods utilizing an array of unimolecular DNA molecules supported on a solid surface. The single molecule including DNA strand regions can form a double-stranded DNA structure that can serve as an in vitro model for double-stranded DNA. The unimolecular DNA-containing molecule, L¹-X¹-L²-X² (where L¹ is the linker to the solid support), folds such that the double-stranded DNA structure forms between X¹ and X², each of which are typically from 6 to 30 nucleotides in length. The '752 patent discloses that L¹, the linker to the solid surface does not have to be nucleotides, but can be polynucleotides or PEG or other molecules with 6 to 50 atoms in the chain leading to the remainder of the molecule. L² is the linker between the two nucleotide-pairing regions. This patent discloses that the linker has a length equivalent to 2 to 4 nucleotides, but can be made of many types of molecular groups, including inter alia alkylene groups of 6 to 24 carbon atoms, polyethylene glycol (PEG), or polynucleotides of 2 to 12 nucleotides in length; preferably PEG or tetraethylene glycol; more preferably 1 to 4 hexaethylene glycols. The disclosures of the '752 patent are incorporated in full herein by reference.

DNA binding research conducted by the Church group has a non-unimolecular system where one DNA-containing molecule is bound to a solid surface, and then a double-stranded DNA structure is generated in situ through primer-initiated oligosynthesis of the complementary strand. Bulyk, M. L., Huang, X. H., Choo, Y. & Church, G. M. (2001) Proc. Natl. Acad. Sci. USA 98, 7158-7163.

The art discloses several studies of the properties of nucleic acid hairpins. The art evaluated the factors affecting the stability of a hairpin loop that causes a nucleic acid molecule to fold back onto itself. Such loops are integral in forming cruciform structures and other structures that act as binding sites or recruiters for molecular binding to nucleic acids for RNA and DNA. An early thermodynamics/nuclear magnetic resonance study of DNA hairpin structures disclosed that 4 T residues in the loop region of a hairpin had increased stability over a loop region with 4 G residues, 4 C residues, or 4 A residues; but that the reason was unclear. Senior, Mary M., Jones, Roger A., & Breslauer, Kenneth J., Proc. Natl. Acad. Sci., USA (1988) 85, 6242-6246. A later study concluded that the identity of the residues at the 5′ end of the loop (the “closing base pair”) have the largest effect on loop stability; hairpin loops being most stable when these residues are closing base pair is a GC base pair. (Moody, Ellen M. & Bevilacqua, Philip C., J. Am. Chem. Soc. (2004) 126, 9570-9577) The size of the single-stranded loop region has somewhat less effect, permitting stable loops from 3 residues to more than 5 residues, with the most reliably stable loop having d(cGNABg), where the loop-closing base pair is in lowercase, “N” is A, C, G, or T, and “B” is C, G, or T. Id.

The study of binding to surface-bound nucleic acids is fraught with technical issues. Proteins, such as potential DNA-binding proteins, tend to adhere to glass surfaces, and in many cases, fail to work in DNA-binding studies with glass in the experimental apparatus. In some cases, the protein has degraded or become improperly folded and is not in the appropriate form to bind DNA as it would in vivo. Blocking agents are known in the art to prevent non-specific binding of proteins to a solid support. Typical known blocking agents include milk, fetal bovine serum, and bovine serum albumin.

The disclosures of the art fail to teach a reliable way to form robust double-stranded nucleic acid structures that are useful in sensitive nucleic acid binding assays. There is a need for re-usable solid-supported double-stranded nucleic acid structures and methods for obtaining sensitive and informative binding information using those structures. There is a need for such materials for applications of the technology to the identification of nucleic acid binding molecules that are an advance over the prior art approaches, such as the SELEX method. There is a need for materials and methods that permit detailed characterization of their nucleic acid binding preferences—particularly sequence specificity. The latter application would be a significant advance over traditional nucleic acid footprinting and mutagenesis gel shift experiments of the prior art for the analysis of sequence specificity. There is a need for materials and methods that permit the detailed study of nucleic acid binding of molecules that do not necessarily affect gene transcription (positively or negatively), a subject matter area in which the prior art has a very limited number of analytical tools available.

### BRIEF SUMMARY OF THE INVENTION

The present invention provides a robust unimolecular double-stranded nucleic acid target for use in nucleic acid-binding assays. The invention improves upon the ground-breaking nucleic acid array technology of the art to provide reliable, reusable materials, system and methods for use in nucleic acid binding assays.

### BRIEF DESCRIPTION OF THE DRAWINGS

In the drawings forming a portion of the description of the invention:

FIG. 1A shows the structure of the Cy3 labeled polyamide, discussed in Example 2, PA1 (Im,Py*,Py,Py,γ,Im,Py,Py,Py,β,Dp, where * denotes the position of the label). The final analysis output was conveniently viewed as shown in FIG. 1B, where the relative size of the bound nucleic acid base letter shows the relative intensity of the signal when the base is at the shown position within X¹, with a three residue anchor sequence of CGC at both ends of X¹. Where two letters are shown, there was intense binding to both of those bases, again with the size showing the relative intensity. For the polyamide studied in this example the preferred DNA cognate site 8-mer was found to be as shown in FIG. 1B. Positions 1 through 8 of the nucleotide binding site show a strong preference for G and T at positions 3 and 4, and C at position 6. A's and T's are equally preferred at each of positions 1, 2, 5, 7, and 8, but are less important than the GT and C. The DNA cognate site motif was selected from sequences in the top Z-score bin (Z>25). A or T is depicted by W; so the consensus binding sequence for the polyamide studied is WWGWWCWW, based on pairing rules. At the bottom of FIG. 1B, a ball-and-stick schematic of the Cy3-polyamide is shown as it is believed to interact with the consensus cognate site shown above it.

FIG. 2A shows the results described in Example 2, the averaged intensities of all of the replicate features for the binding of the assay molecule PA1 to the 8-position sequence variation DNA target array. Statistical Z-scores are shown at the marked arrows. FIG. 2B shows a graph of the correlation between CSI target array binding results intensities and Kₐ as determined from nuclease protection experiments.

FIG. 3 shows a graph of the abundance of each sequence motif in each Z-score bin for the sequence preferences of the polyamide assay molecule discussed in Example 2, where W represents A or T. From this graph, the strong preference for WWGWWCWW within the variable sequence portion of X¹ is evident.

FIG. 4 shows array binding data from the target array binding assay for assay molecule PA1, discussed in Example 2. FIG. 4A shows a correlation plot of the target array binding assay (intensity versus intensity) for two of the four CSI microarray replicates. Intensities have been normalized and background subtracted so that the mean intensity is zero. Average correlation value between replicates is 0.88. The diagonal line represents perfect correlation. FIG. 4B shows a plot of the end-flanking nucleotides for positions 1 and 8 (N1, N8) of the variable 8-mer portion of the PA1 consensus sequence. It is a plot of the target array binding intensity of a W (A or T) versus an S(C or G). The intensities of all features that contain any permutation of the core consensus sequence with the indicated flanking sequence are averaged together.

FIG. 5 shows the PA1 array binding data analyzed through the generation of a molecular recognition landscape, where the highest intensity is graphed at the center for all 8 preferred nucleotides, and then each variation from the consensus drops the datapoint out to a farther-out concentric ring.

FIG. 6 shows the CSI assay data for Exd, as discussed in Example 3. FIG. 6A shows the crystal structure of Exd bound to DNA. The dotted line represents residues that are disordered in the crystal structure. The Exd residue R2C, is the unstructured amino acid to which the Cy3 dye is attached. FIG. 6B shows a graph of the dependence of fluorescence intensity of Exd-DNA binding upon the number of consecutive G nucleotides. FIG. 6C shows the results from electrophoretic mobility shift assays of G-rich and Consensus hairpin DNA sequences with Exd and polyamide. Consensus DNA bears a composite Exd-polyamide binding site and shows a band shift upon addition of these two molecules. The G-rich DNA in the absence of Exd has a band (bottom arrow), which is higher than the Consensus DNA shift with Exd and polyamide. This band is completely shifted (top arrow) upon addition of Exd, but is unaffected by polyamide. FIG. 6D shows the results of an electrophoretic mobility shift assay of G-rich and Consensus hairpin DNA with increasing concentrations (0.15, 0.3, 0.625, 1.25, 2.5, and 5 μM) of PIPER (N,N-Bis[2-(1-piperidino)ethyl]-3,4,9,10-perylenetetracarboxylic diimide), a compound that stabilizes G-quadruplexes. In the presence of 5 μM of PIPER, G-rich DNA shows significant aggregation (as evidenced by the immobility from the well) while Consensus DNA does not.

FIG. 7 shows a schematic for a DNA hairpin target array according to the present invention with all permutations of an 8 nucleotide sequence and its complement within X¹ and X², flanked by a three base pair anchor sequence at both ends, a four-residue linker L² (here TCCT), and the linker L¹ to the solid support. The schematic shows an array with feature clusters with a sample intensity output from a binding assay with an assay molecule.

7 4 FIG. 8 shows CSI profile data for PA2 and PA3 with Exd, as described in Example 3. FIG. 8A) Left: Structures of polyamide-peptide conjugates PA2 and PA3 (ImImPy*Py-γ-ImPyPyPy-β-Dp). The expected DNA-binding sequence is 5′-WGWCCWW-3′ based on the ring pairing rules for polyamides (Pandolfi, P. P. (2001) Oncogene 20, 3116-3127). The peptide sequence, N-FYPWMK-C, is conjugated to Py*. Right: Schematic of cooperative binding of polyamide and Exd to DNA. FIG. 8B) Logos for the main motifs found in the CSI profile for PA2-Exd (left) and PA3-Exd (middle) using motif-finding algorithms (Bailey, T. L. & Elkan, C. (1994) Proc. of the 2nd Intl. Conference on Intelligent Systems for Mol. Biol., AAAI Press, Menlo Park, Calif., 28-36; Hughes, J. D., Estep, P. W., Tavazoie, S. & Church, G. M. (2000) J. Mol. Biol. 296, 1205-1214; Liu, X. S., Brutlag, D. L. & Liu, J. S. (2002) Nat. Biotechnol. 20, 835-839). Logos are based on sequences from the top Z-score bin (Z>5.0). Right: Representation of expected binding orientation of Exd and polyamide in the motif. Boxes indicate the binding position of Exd and polyamide in the sequence. An underline instead of a box indicates that the polyamide is binding in an inverted orientation. FIG. 8C) Plot of the relative abundance of each sequence motif in each Z-score bin. Left: PA2 with Exd. Right: PA3 with Exd.

FIG. 9 shows solution binding and molecular modeling data for the binding of the cooperative Exd polyamide complex with the DNA used in the target CSI array. FIG. 9A) Electrophoretic Mobility Shift Analysis (EMSA). Top Row: 50 nM PA2 incubated with increasing concentrations of Exd (in nM). Bottom Row: 50 nM PA3 with an Exd titration. Labels above each pair of EMSAs indicate the binding motif used. Below each pair of EMSAs are the sequences used. Boxes indicate the Exd and polyamide binding sites. An underline instead of a box indicates that the polyamide is binding in an inverted orientation. FIG. 9B) Molecular modeling (Schneider, T. D. & Stephens, R. M. (1990) Nucl. Acids Res. 18, 6097-6100; Berman, H. M., Westbrook, J., Feng, Z., Gilliland, G., Bhat, T. N., Weissig, H., Shindyalov, I. N., Bourne, P. E. (2000) Nucl. Acids Res. 28, 235-242) images of Exd and polyamide bound in Consensus, Consensus+1, Consensus−1, and Inverse orientations. Models are based on aligning the DNA from the protein database (pdb) files 1B8I and 1M18 (Berman, H. M., Westbrook, J., Feng, Z., Gilliland, G., Bhat, T. N., Weissig, H., Shindyalov, I. N., Bourne, P. E. (2000) Nucl. Acids Res. 28, 235-242). Distances are calculated from the N-methyl group of the analogous ring to which the linker is connected on PA2 and PA3 to the carboxyl carbon of the methionine of the recruitment peptide (FYPWM) bound to Exd in the crystal structure. FIG. 9C) Table listing the K_{D} calculated from the EMSA and the fluorescence extracted from the CSI profile for each polyamide-Exd complex.

### DETAILED DESCRIPTION OF THE INVENTION

Cognate Site Identifier Technology. To bridge the gap in the art between computational methods and molecular recognition properties of nucleic acid-binding molecules, the present invention provides a high-throughput platform that can rapidly and reliably provide information about binding to the cognate sites (binding sites) of nucleic acid-binding molecules. In a preferred embodiment, this platform utilizes a unimolecular double stranded nucleic acid array that displays all possible permutations of a nucleic acid bases in a target sequence of a given length. For example, the sequence-related binding affinity of a molecule to a DNA target array can be obtained for a target array that has all permutations of an 8 base pair target sequence (32,896 molecules).

The disclosures of this work by the present inventor are published at Proc. Natl. Acad. Sci., USA, 104(4), 867-872 (2006), and are incorporated in full herein by reference.

Also disclosed are methods for a systematic approach to treat the array data that can be applied to arrays of greater complexity. These include computational methods. Since most metazoan DNA binding proteins target 6-10 base pairs (Wingender, E., Chen, X., Fricke, E., Geffers, R., Hehl, R., Liebich, I., Krull, M., Matys, V., Michael, H., Ohnhauser, R., et al. (2001) Nucleic Acids Res. 29, 281-283) and DNA binding small molecules rarely exceed 8 bp (Neidle, S. (2001) Nat. Prod. Rep. 18, 291-309), the cognate site identifier (CSI) arrays of the present invention are useful in methods for identifying and ranking sequences preferred by a nucleic acid binding molecule (also called a “ligand”) by itself, or in cooperatively binding pairs. The approach derives binding profiles from a rapid, unbiased, and unsupervised examination of the entire nucleic acid sequence space.

For example, in one embodiment, a single array according to the present invention includes every possible combined variation of each of ten sequence positions. In this manner, the present invention can avoid the data bias problem encountered in other methods of the art, that were limited to a nucleic acid target pool generated from a certain tissue or maturation stage. The invention contemplates application of the arrays and analyses to nucleic acid binding proteins from any organism and in the case of small molecules—utility in the prediction of binding sites in any genome. The invention additionally contemplates embodiments in which combinatorial selection of sequences is utilized in place of including every possible combined variation of nucleic acid residues.

Methods of the art are unable to define the range of sequences recognized by nucleic acid binding molecules. The present inventor's approach is useful in determining the specificity of nucleic acid binding molecules. In this approach, the comprehensive sequence recognition landscape of nucleic acid binding molecules is determined in a rapid, unbiased, and unsupervised format. Due to the ability of the present invention to display of an entire sequence space (within a certain size), there is no limitation on the use of proteins of a specific organism or a specific class of small nucleic acid binding molecules.

The present invention permits a comprehensive mutational analysis in a single experiment by enabling examination of the entire sequence space at once. The invention thus provides information on the contribution of each nucleotide residue to the molecular recognition event between the nucleic acid binding molecule and its cognate site(s). Moreover, the system permits query of nucleic acid binding preferences of the nucleic acid binding molecules (e.g. proteins or small molecules) under identical conditions. The inventors contemplate that the accumulation of binding data from CSI analysis of different molecules will lead to the elucidation of the molecular recognition by a cluster of residues displayed on the surface of nucleic acid binding molecules. It is hoped that such modular perspective will be helpful in the art to coherently decipher the principles of molecular recognition displayed by nucleic acid binding molecules.

By determining the complete sequence recognition profile of any nucleic acid binding molecule, the CSI array analysis also bridges the gap between the ChIP-chip approach and bioinformatic approaches of identifying regulatory nucleic acid elements in genomes. For example, from a single CSI array experiment one can unambiguously validate (and order by affinity) all binding sites identified by ChIp-chip assays. Furthermore, the rank-order of the sequences is useful for computationally mining the genome for possible binding sites that are missed by other methods. The CSI array analysis enables a coherent analysis of transcriptome studies by scanning for the presence of a range of possible binding sites in co-regulated genes. Application of a CSI-analysis in conjunction with other approaches is useful for reducing the discrepancies between and absence of discernible binding sites in co-regulated genes or the inability to detect protein binding at biologically relevant sites in vivo by ChIP-chip analysis.

The CSI array also has pharmaceutical and diagnostic utility. It provides a much-needed high-throughput approach for the design and development of novel classes of sequence-specific nucleic acid binding molecules. It is a useful method for screening for molecules that target disease-causing DNA binding proteins, and useful for or to identify changes in regulatory or nucleic acid binding proteins in cells or tissues.

As the ability to display more oligonucleotide features on a surface increases, the CSI approach is routinely scalable to represent larger sets of sequence variants. The invention contemplates nucleic acid target variable regions from 6 to 60 nucleotides in length. Research to date has shown that a great many metazoan DNA binding proteins interact directly with a 6 to 10 nucleotide long region of nucleic acid, so an embodiment of the array presenting all variants of a 10 nucleic acid residue sequence in X¹ and X² would be very useful and may be all that is required. The longer targets are useful, for example, in assays of nucleic acid binding of molecules with multiple binding domains or cofactors, where the binding complex interacts with more than one region of the target nucleic acid at the same time. The present invention provides a powerful new tool for tackling the important challenge of deciphering the nucleic acid recognition code of nucleic acid binding molecules, individually or in cooperatively assembling complexes.

The present invention relates to compositions and methods for comprehensive profiling of the binding properties of a nucleic acid binding molecule. In particular, the present invention relates to a method for performing molecular interaction assays on solid surfaces as well as pretreatments (e.g. coatings) for controlling non-specific adsorption.

In some embodiments, the present invention provides a product comprising an array of oligonucleotides bound to a surface where the bound oligonucleotides are unimolecular nucleic acid molecules. An assay molecule (e.g. DNA ligand) is applied to the target array surface to probe the binding affinity and specificity of the assay molecule to each particular oligonucleotide arrayed on the surface. In some embodiments, these unimolecular oligonucleotides form B-form hairpins or other non-B-form structures with themselves. In this way, unimolecular oligonucleotides self-anneal and form double-stranded and other structures that nucleic acid binding molecules interact with in a sequence-specific manner.

In some embodiments, the array of target molecules is comprised of unimolecular DNA. In some of those embodiments, the signal molecules contain at least eight base pairs. In some of those embodiments, the single stranded DNA assumes hairpin structures, including B-form DNA duplexes, abnormal structures such as cruciforms, mismatched bubbles, and bulges.

The inventions described herein are useful for any number of analyses wherein a nucleic acid binding molecule interacts with an array of oligonucleotides, such as in determining binding affinities, measuring the binding effects of short-range secondary structure in nucleic acids, etc.

The arrays of the invention have an additional advantage in that rudimentary data clustering can be structured in the array by building an array wherein islands of nucleic acids differ systematically (e.g. by length or primary sequence). The interactions of any given nucleic acid sequence for any given analyte can be quickly and exhaustively investigated. Likewise, the effects of short-range secondary structure in nucleic acids can be investigated by building an array wherein the islands of nucleic acids differ in sequence such that the islands contain nucleic acid sequences which progressively contain more stable secondary structures and then scanning the array after exposure to a given analyte.

Assay molecule. The assay molecule is the molecule that is being investigated for its ability to bind to nucleic acid. Typically, the assay molecule will be a DNA-binding protein or small molecule.

Target. The target is the nucleic acid-containing molecule that is being investigated for binding by the assay molecule. In a typical array assay, there are multiple targets bound to a solid surface. In a preferred embodiment of the present invention, the target is a unimolecular nucleic acid-containing molecule, L¹-X¹-L²-X² where L¹ is the linker to the solid support, X¹ and X² are nucleic acid containing regions that form a double-stranded structure when the molecule folds, and L² is a folding link region between the two nucleotide-pairing regions.

L¹. The present inventors found that the linker of the nucleic acid target to the supporting surface can be significantly longer than suggested in the art. Preferably, L¹ is a polynucleotide of ribonucleotide or deoxyribonucleotide residues that may contain nucleotide analogs or chemically modified nucleotides. The length of L¹ is preferably 8 to 30 nucleotides in length (therefore having about 56 to 210 atoms in the polynucleotide backbone chain), more preferably from 10 to 20 nucleotides in length (about 70 to 140 atoms in the polynucleotide backbone chain). The present inventors typically use a 15-mer polynucleotide, which is more than 100 atoms in length of the polynucleotide backbone chain. Preferably, the L¹ sequence does not include the stable hairpin sequence (GNA) preferred for L², below. The present inventors surprisingly found that neither the additional conformational mobility of the surface-bound DNA-containing molecules nor the concomitantly increased chance of interference between molecules in a physically close (dense) array setup resulting from the increase in L¹ linker length negatively affect or skew the DNA binding results.

The array density can still be very high with this long linker. The present inventors have had success with more than one million unimolecular targets in a single array and approaching two million in a single array with an L¹ linker more than 100 atoms in length.

X¹ and X². X¹ and X² are made up of nucleic acid residues, including DNA or RNA residues, chemically-modified residues and residue analogs. The nucleic acid residues in the X¹ and X² regions will interact with each other when the target molecule folds to make a nucleic acid structure (DNA or RNA) with double-stranded regions, including hairpins, cruciform structures, bulges, bubbles, mismatches, B-form helical or other double-stranded structures.

A typical DNA-binding protein interacts with a single turn of a helix (about 10 DNA bases) or a pair of adjacent turns. However, in some cases, DNA-binding proteins and co-factors form a multimeric complex that can interact with DNA in vivo at more than one site simultaneously. To assay nucleic acid binding by those type of macromolecular structures, a combined target DNA cognate site may be longer than 8 to 10 nucleotides.

The nucleic acid-containing portions of the target are each from 4 to 100 nucleotides in length, for monomeric assay molecules preferably 6 to 30 nucleotides, and more preferably 15 to 26 nucleotides; for multimeric assay molecules, more preferably 15 to 50 nucleotides. The nucleotide sequences of X¹ and X² are complementary to one another. Preferably, X¹ and X² have sequences as desired by the experiment. In some embodiments, X¹ and X² are cases perfectly complementary to one another (100% complementary). In some embodiments, X¹ and X² will form standard B-form DNA double helices. In some embodiments, X¹ and X² are not 100% complementary, and have mismatches or bulges or other structures form. In these cases, the complementarity is as is necessary for the desired target tertiary structure, ranging from 75% complementarity (typically with shorter sequences for X¹ and X², where a single base change will give rise to a high decrease in percent complementarity) upward to 100%. The residues that base pair between X¹ and X² are referred to herein as the “corresponding residues”. This language is introduced to accommodate the cases of non-standard structure, for example where there is a bulge that causes a misalignment between X¹ and X² such that multiple base-pairing regions are not fully consecutive, and once the residues are base-pairing again, they are corresponding. In some embodiments, X² is shorter than X¹, and in calculating percent complementarity, only those residues of X¹ that correspond to residues in X² should be included in the calculation.

Double-Strand Region Anchor. Preferably, the DNA-containing portions of the target form anchor regions of strongly binding bases (e.g. G=C) at both ends of the double-stranded portion. These relatively strong bonds at each end of the region that is desired to be double-stranded have been found in practice to provide an important service in enhancing reliability in the formation of double-stranded structure through the desired assay target region. In typical assays, it is preferred that these residues are NOT varied but rather are constant throughout in the array, except in the controls to determine the effect of the anchor region on the assay. One anchor region is at the open end of the unimolecular double-stranded structure, therefore at the end of X¹ near L¹ (the 3′ end of X¹), and at the end of X² furthest from L² (the 5′ end of X²). Another anchor region is at the end of X¹ near L² (the 5′ end of X¹) and the end of X² closest to L² (The 3′ end of X²). The anchor region is preferably from 1 to 3 nucleotides in length; so that the inclusion of two anchor regions define from 2 to 6 of the nucleotides of the preferred 15 to 26 nucleotide length of X¹ and X². In a preferred embodiment, the 5′ end of X¹ is a C residue, and the 3′ end of X² is a G residue.

In some cases, a nucleic acid is bound by a protein or protein complex in adjacent sites. The present invention has been used to assay binding within such a complex by building a split site, which had an anchor sequence, a variable region, a constant region, and a variable region and another anchor sequence, so that there were two distinct sites being studied. Each site was for a different nucleic acid binding protein that interact with each other; or for two domains of one with an adjacent major groove binding. They may also be interacting nucleic acid-binding monomers or interacting domains.

L². L² is the linker between the two nucleotide pairing regions. The disclosures of the '752 patent teach a length of 2 to 4 nucleotides, but that this linker can be made of many types of molecular fragments. The present inventors have found that it is best that the linker is 3 nucleotides having the sequence GNA, where N is any nucleotide residue A, C, G, or T. GNA which was disclosed in the literature to be a DNA loop-stabilizing motif. (Moody, Ellen M., Bevilacqua, Philip C., J. Am. Chem. Soc. (2004) 126, 9570-9577)

Blocking. A step to prevent non-specific blocking of the assay molecule to the DNA structure is a key step in properly-functioning binding. First, a control of the assay molecule in binding to a blank solid support should be conducted using various blocking agents to determine the best blocking agent for that particular assay molecule and experimental conditions. Typical blocking agents that should be included in the blocking control are milk (e.g. 2% liquid milk from a grocery store, powdered milk from a grocery store); fetal bovine serum (FBS); bovine serum albumin (BSA); E. coli extract from DH5α coli; mixtures containing free DNA as a background binder (e.g. commercially available calf thymus DNA, salmon sperm DNA, poly dIdC, tRNA).

Surface coating can also prevent background binding. Several proprietary coatings are commercially available and have been found useful, e.g. PEGylated surface or alkylated surface (GenTel BioSciences, Inc., Madison, Wis.). Also widely commercially available are compounds for pre-treated the surface to deter non-specific protein binding, such as silanizing agents (for silanizing surfaces), and sugars such as amylose, which coat to some degree and also may provide conditions for optimal protein binding.

We have found that it is not always necessary to remove excess blocking agent, as in a “wash” step. Typically, a DNA binding array surface will be treated with silane or PEG and then excess reagent washed off. However, in the case of non-specific competitors (e.g. a milk blocking agent), the experiment can be conducted in the presence of the blocking agent. Thus, a useful method of the invention will preferably include the following steps: (i) coat and (iii) assay; or (i) coat, (ii) wash and (iii) assay.

Protein Binding Optimization. Experimental conditions should be routinely investigated for each new protein/nucleic acid system being studied. Typical conditions that should be explored are well known in the nucleic acid-binding art and include various salt and buffer concentrations, temperature, assay molecule concentrations.

Life Science Applications: One embodiment of the present invention provides a method of determining the binding preference of a DNA binding protein. In an example of this embodiment, the protein is known and the sequence preferences of that protein are sought. The method comprises first presenting a solid surface bearing an array of oligonucleotides of every possible sequence. The solid surface is then brought into contact with a DNA binding protein. In a preferred embodiment of this method, these DNA binding proteins are recombinant proteins, purified proteins, or a combination thereof. Once the DNA binding protein assay molecule(s) are brought into contact with the array, the pattern of binding is then detected using fluorescence or some other standard biomolecular detection method known in the art or later developed. The detection signal is then quantified to thereby deduce the affinity of the DNA binding molecule (ligand) for its specific target nucleic acid in the sample tested.

In some embodiments, materials and methods of the invention are used in the design or engineering of DNA binding proteins, including mutants of endogenous proteins that bind DNA in a sequence-specific manner.

In further embodiments, the invention provides a method to characterize putative, annotated proteins identified by informatic analysis of recently sequenced genomes, including human. In an example of this embodiment, the putative protein is not known and the sequence preferences of that protein are sought. Because sequence specificity of these putative, annotated proteins cannot be reliably predicted, it must be determined empirically. The method comprises expressing coding sequences of the annotated protein as a recombinant polypeptide. Next, a solid surface bearing an array of oligonucleotides with the desired sequence variation (e.g. all variants of a 10-mer) is constructed. The solid surface is then brought into contact with the expressed polypeptide. The recombinant polypeptides are brought into contact with the array, the pattern of binding is detected using a molecular detection method, preferably biomolecular. The detection signal is then quantified to rapidly determine the comprehensive sequence preference of the polypeptides. In another example of this embodiment, combinations of recombinant polypeptides are applied to the same array to determine specificity of cooperatively binding complexes.

A further embodiment provides a tool to validate, or refine results acquired by recently developed method (such as ChIP-chip methodology) to determine sequence preference of a DNA binding protein in a cell. In this embodiment, the DNA binding protein is known and the sequence preferences of that protein have been roughly approximated using ChIP-chip methodology but need to be refined and validated. The method comprises first presenting a solid surface bearing an array of DNA duplexes that span the entire roughly defined chromatin region. The arrayed sequences are brought into contact with a DNA binding protein such that the targeted sequence(s) are identified. These DNA binding proteins can be recombinant or purified proteins, or combinations thereof. Once the DNA binding proteins are brought into contact with the array, the pattern of binding is then detected using fluorescence or some other biomolecule detection method. The detection signal is then quantified to thereby deduce the affinity DNA binding molecule (ligand) for its specific target nucleic acid in the sample tested. This new high-throughput method replaces cumbersome methodologies currently used to validate ChIP-chip data such as quantitative PCR and electrophoretic mobility shift assays.

Drug Discovery Applications: One embodiment of the present invention provides a method of screening compounds that could serve as potential therapeutics, including small molecules. In this embodiment, the small molecule is known and the sequence preference of the small molecule(s) is sought. The method comprises first presenting a solid surface bearing an array of oligonucleotides of every possible sequence. The solid surface is then brought into contact with a small molecule or mixtures of small molecules. These small molecules can be synthetic or natural products, or combinations thereof. Once the small molecules are brought into contact with the array, the pattern of binding is then detected using fluorescence or some other biomolecule detection method. The detection signal is then quantified to thereby deduce the affinity of the small molecules for its specific target nucleic acid in the sample tested.

In some embodiments, the method is used to identify compounds that can disrupt diseases caused by aberrant binding between DNA and proteins. In some embodiments, the method is used to screen for molecules that target disease-causing DNA binding proteins or to identify changes in regulatory or nucleic acid binding proteins in cells or tissues.

Diagnostic Applications: The present invention also provides a method of diagnosing a disorder through a specific DNA binding pattern. In some embodiments, a healthy or diseased condition can be identified or correlated with a specific binding pattern. These binding patterns are also called fingerprints. In this way, the tool is useful for predictive or diagnostic purposes. The method comprises first presenting a solid surface bearing an array of oligonucleotides of every possible sequence. The solid surface is then brought into contact with a sample. The sample can be a tissue sample, a cell extract or some other sample taken from a patient. In some cases, these samples are labeled using a covalent method to apply a fluorescent or other tag to a sample. Once the sample is brought into contact with the array, the pattern of binding is then detected using fluorescence or some other biomolecule detection method. The detection signal is quantified to identify a specific pattern of binding and then determined whether the signal correlates with a healthy developmental, differentiated, or diseased state(s).

In a further embodiment, a specific nucleic acid binding pattern is further examined using molecular probes, such as antibodies, that recognize specific proteins and/or post-translational modifications of these proteins. These patterns can be further correlated with disease state, function, or cell differentiation.

In some embodiments, the tissue sample is a whole cell extract, a nuclear extract, a clarified extract, or extract enriched for nucleic acid binding proteins. In some embodiments, all proteins in a tissue sample are labeled using a fluorescent dye.

### EXAMPLES OF THE INVENTION

The methods disclosed herein are useful in the study of transcription factors and their regulatory role in gene expression, and also to determine the relative effectiveness of synthetic molecules that mimic natural transcription factors or counteract the action of malfunctioning factors. There is a need for tools to rapidly study artificial transcription factors, which will lead to identification and determination of how to control gene networks that govern cell fate, and in studying the value of ATFs as precision-tailored therapeutic agents.

### Example 1

### Manufacture of a DNA Array

Duplex DNA Microarrays were synthesized using a Maskless Array Synthesizer (MAS) (Singh-Gasson, S., Green, R. D., Yeu, Y., Nelson, C., Blattner, F., Sussman, M. R., Cerrina, F. (1999) Nat. Biotechnol. 17, 974-978). Homopolymer (T₁₀) linkers were covalently attached to monohydroxysilane glass slides. Oligonucleotides were then synthesized on the homopolymers to create a high-density oligonucleotide microarray.

The array surface is derivatized such that the density of oligonucleotides is sufficiently low within the same feature no one oligonucleotide should hybridize with its neighbors. Four copies of every variation of an 8 nucleotide sequence with three anchor residues at each end (X¹) required a total of 131,584 features per array. Each array also has a distinct “reference” sequence synthesized at the edges for quality control and to align the grid.

Hairpin Formation Percentage. In two distinct features on the array two sequences were presented: one that forms a hairpin (5′ CGC-TTAGTTCA-CGC-TCCT-GCG-TGAACTAA-GCG 3′; where the underlined portions are X¹ and X², respectively) and one that does not (5′ CGC-TTAGTTCA-CGC 3′; which would be like X¹, alone). Using a Cy3 labeled DNA target that is complementary to the core sequence present in both oligonucleotides (5′ GCG-TGAACTAA-GCG 3′; which would be like X², alone) the ability of the complementary strand to bind the hairpin is determined versus the single stranded DNA molecules. The fluorescence intensity of the hairpin sequence is divided by the fluorescence intensity of the single-stranded sequence. The averaged background-subtracted intensity ratio of the double-stranded versus the single-stranded features indicated 95.6% hairpin formation.

### Example 2

### Array Binding Assay of an Engineered Small Molecule

To demonstrate the accuracy and fidelity of a nucleic acid array of the present invention, we used a polyamide engineered to target a specific DNA sequence to generate an artificial small molecular transcription factor. The nucleic acid sequence binding preferences of polyamides are well-known in the art, so there is a benchmark against which to compare the nucleic acid binding data from this example of a CSI array of the present invention.

Transcription factors play a primary regulatory role in gene expression and thereby in defining cell fate. Transcription factors also perform a central role in regulating cellular physiology and homeostasis. Different signaling pathways instruct specific transcription factors to regulate expression of target genes. Inappropriate action of transcription factors, either due to aberrant signaling or to mutation in the factor themselves, is linked to the onset of a wide array of diseases including developmental defects, immune disorders, cancer, and diabetes.

A major challenge at the interface of chemistry, biology and molecular medicine is the ability to design synthetic molecules that mimic natural transcription factors or counteract the action of malfunctioning factors. Artificial transcription factors (ATFs), by virtue of their ability to regulate targeted genes, will serve as powerful tools to identify and control gene networks that govern cell fate. The ultimate value of ATFs lies in their potential application as precision-tailored therapeutic agents.

### A. Modular Design of Transcription Factors and Artificial Transcription Factors

To design small molecule ATFs, we mimicked the domain architecture of natural transcription factors. Typical transcription factors bear at least two modules: (i) a DNA binding domain that permits them to target specific genes, and (ii) a regulatory domain that confers the ability to control the expression of the targeted gene. This modular structure of natural factors lends itself to modular reconstruction using synthetic counterparts. The DNA binding domain was replaced with sequence-specific polyamides; the regulatory domains were replaced with non-natural regulatory modules that can either activate or repress transcription. In essence, we use polyamides as a scaffold to deliver functional moieties to specific sites in the genome.

Polyamides satisfy several criteria: (i) they have well-understood design rules to permit creation of a polyamide that targets any desired 6-8 base pair DNA sequence; (ii) polyamides bind the minor groove of DNA without perturbing its structure and can bind DNA wrapped on a nucleosome; (iii) polyamides display high affinity for DNA; (iv) polyamides are readily synthesized using robust solid phase methods; and (v) polyamides can be conjugated to additional regulatory molecules or domains.

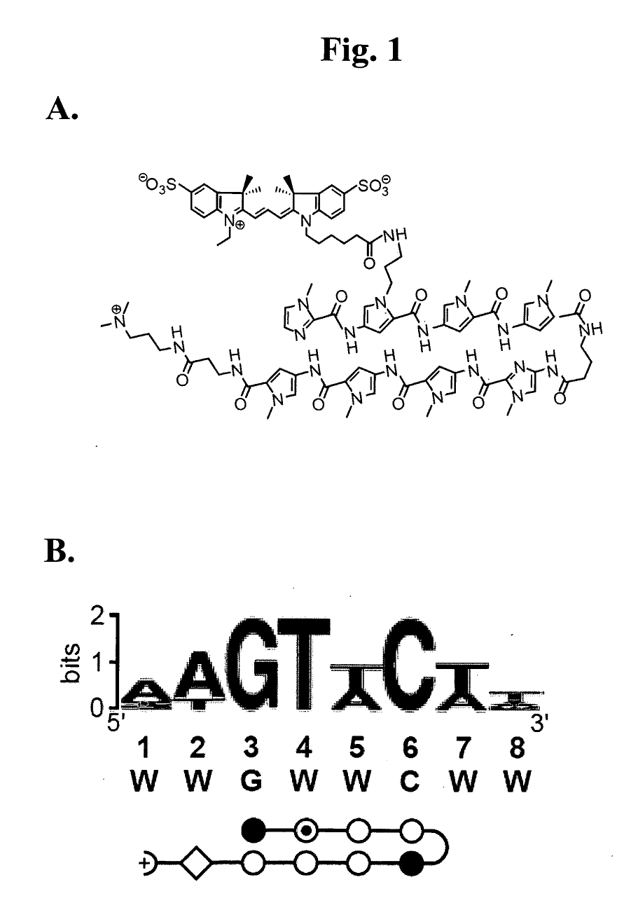

Polyamide Synthesis: Polyamide-Cy3 conjugate PA1, shown in FIG. 1A (Im,Py,Py,Py,γ,Im,Py,Py,Py,β,Dp) was prepared employing an orthogonally protected N-(phthalimidopropyl)pyrrole building block in standard Boc-based solid phase synthesis (Baird, E. E. & Dervan, P. B. (1996) J. Am. Chem. Soc. 118, 6141-6146). Cleavage of the polyamide from PAM resin (100 mg) by treatment with dimethylaminopropylamine (1 mL) also removed the phthalimide protecting group to give the free base. The crude cleavage mixture was diluted with 0.1% TFA (aq) and acetonitrile to a final volume of 5 mL and loaded onto a preconditioned solid phase extraction column (C₁₈ bonded phase). After washing with a 4:1 (v:v) solution of 0.1% TFA (aq) and acetonitrile, product was eluted with methanol and azeotroped from toluene to afford the aminopropyl precursor of PA1 as a slightly yellow solid. The identity and purity of this intermediate was verified by analytical HPLC and MALDI-TOF MS and it was used without further manipulation.

The intermediate free base (0.5 μmol) was dissolved in anhydrous DMF (0.45 mL) and DIEA (0.05 mL). To this solution was added a pre-packaged amine-reactive Cy3 fluorophore (1 mg). The resulting mixture was agitated in the absence of light, at ambient temperature, for 4 hours. Cy3 fluorophores were obtained as succinimidyl esters from Amersham and used as received. Crude products were purified by preparative HPLC using C₁₈ bonded phase silica with 0.1% TFA and acetonitrile as mobile phases. The purity and identity of product was confirmed by analytical HPLC and MALDI-TOF MS. PA1 was labeled as shown in FIG. 1A: Im,Py*,Py,Py,γ,Im,Py,Py,Py,β,Dp, where * denotes the position of the label. UV-Vis (H₂O) λₘₐₓ nm (εM cm⁻¹): 313 (69,500), 555 (75,000). MALDI-TOF MS (monoisotopic) [M+H] 1877.60 (1877.81 calculated for C₉₁H₁₁₂N₂₄O₁₇S₂).

### B. ATFs are Potent Stimulators of Transcription

The ATFs made for this example were shown using methods of the art to bind to targeted sites, recruit the multicomplex transcriptional machinery to the promoter and lead to expression of a proximal gene. Transcription assays using varying amounts of the ATFs and incorporating radiolabeled nucleotides show the relative transcriptional activation of the target gene.

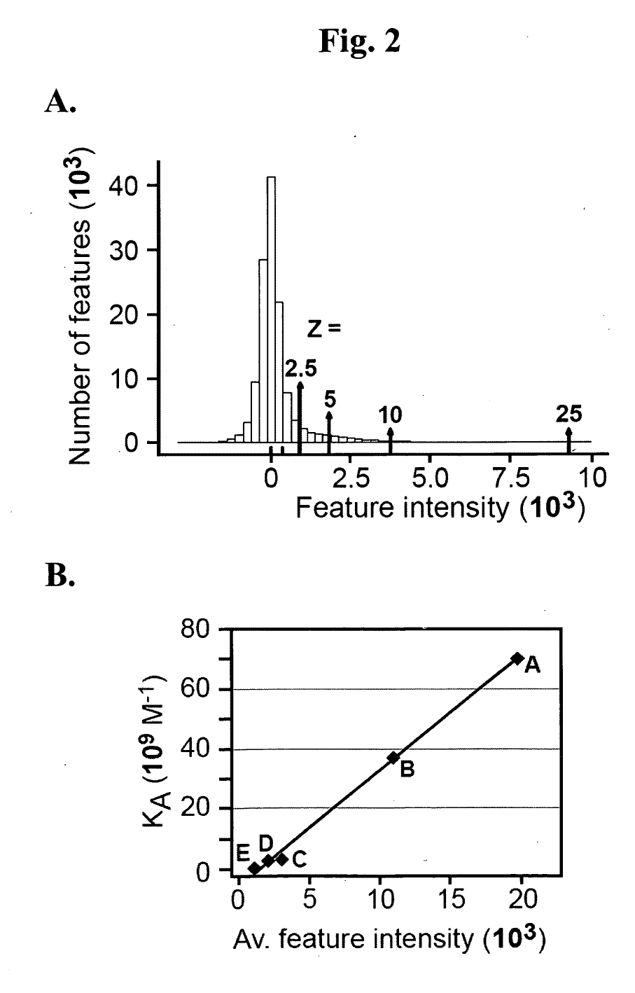

Polyamides were engineered to target specific DNA sequences (e.g. PA1, shown in FIG. 2A). Polyamides are DNA binding small molecules composed of N-methylpyrrole (Py) and N-methylimidazole (Im) heterocycle rings. The arrangement of the heterocycles (Im or Py) can be programmed to create polyamides that target most naturally occurring 6 to 8 base pair DNA sequences (Pandolfi, P. P. (2001) Oncogene 20, 3116-3127). PA1 (ImPy*PyPy-γ-ImPyPyPy-β-Dp), in particular, was designed to target the sequence 5′-WWGWWCWW-3′ (W=A or T) (Trauger, J. W., Baird, E. E. & Dervan, P. B. (1996) Nature 382, 559-561). A Cy3 fluorescent dye is conjugated to the N-methyl position of an internal pyrrole (Py*). Such conjugation does not meaningfully alter the DNA binding properties of the polyamides (Rucker, V. C., Foister, S., Melander, C. & Dervan, P. B. (2003) J. Am. Chem. Soc. 125, 1195-1202). Previous solution based footprinting (Trauger, J. W., Baird, E. E. & Dervan, P. B. (1996) Nature 382, 559-561) and dye displacement assays (Kim, Y., Geiger, J. H., Hahn, S. & Sigler, P. B. (1993) Nature 365, 512-520) have shown that polyamides discriminate very highly between their targeted cognate site and sites that differ by a single base pair. Thus, PA1, a well characterized DNA binding molecule, provides an example of the ability of the CSI array to accurately identify its sequence recognition landscape.

### C. Cognate Site Identifier Array Analysis of a Polyamide

A cognate site identifier (CSI) array is a high-throughput platform useful for the identification and ranking of sequences preferred by a DNA binding molecule. The CSI method permits the determination of binding profiles from a rapid, unbiased, unsupervised examination of the entire DNA sequence space (10 positions of sequence variation within X¹ and X² in this example, in addition to 3 base pair anchor regions at each end) under identical reaction conditions.

Hairpin Target CSI Microarray. Each hairpin target was composed of a central permuted 8 base pair region with X¹ with a three base pair CGC sequence anchor region flanking either end, with a complementary sequence on X². A fluorescently tagged nucleic acid binding assay molecule was applied to the microarray to generate the nucleic acid binding profile for that assay molecule. Analysis of the array signal intensity gives rise to a reference grid showing the binding intensity output for the array. High intensity features indicate tight binding of the assay molecule to that specific target sequence.

Binding Assay: Microarray slides were immersed in 1×PBS (Phosphate Buffered Saline) and placed in a 90° C. water bath for 30 minutes to induce hairpin formation of the oligonucleotides. Slides were then transferred to a tube of non-stringent wash buffer (Saline-Sodium Phosphate-EDTA Buffer, 0.01% v/v Tween-20) and scanned to check for low background (<200 intensity). Microarrays were scanned using a ScanArray 5000 and the image files extracted with GenePix Pro version 3.0.

Polyamide binding: Microarrays prepared as above were placed in the microarray hybridization chamber and washed twice with non-stringent wash buffer. Polyamide was diluted to 5 nM in Hyb buffer (100 mM MES, 1 M NaCl, 20 mM EDTA, 0.01% v/v Tween-20). Polyamide (5 nM) was then added to the hybridization chamber and incubated at room temperature overnight (16 hours). Finally, the microarrays were washed twice with non-stringent wash buffer and scanned.

Protein Binding: The microarrays were washed with 150 mM K-glutamate, 50 mM HEPES pH 7.5, 5% glycerol (reaction buffer), for 5 minutes. Cy3-labeled Exd and polyamide were diluted in reaction buffer to a final concentration of 20 nM and 50 nM, respectively. This solution was added to the hybridization chamber and incubated for 30 minutes. Subsequently, the microarrays were washed with reaction buffer and scanned.

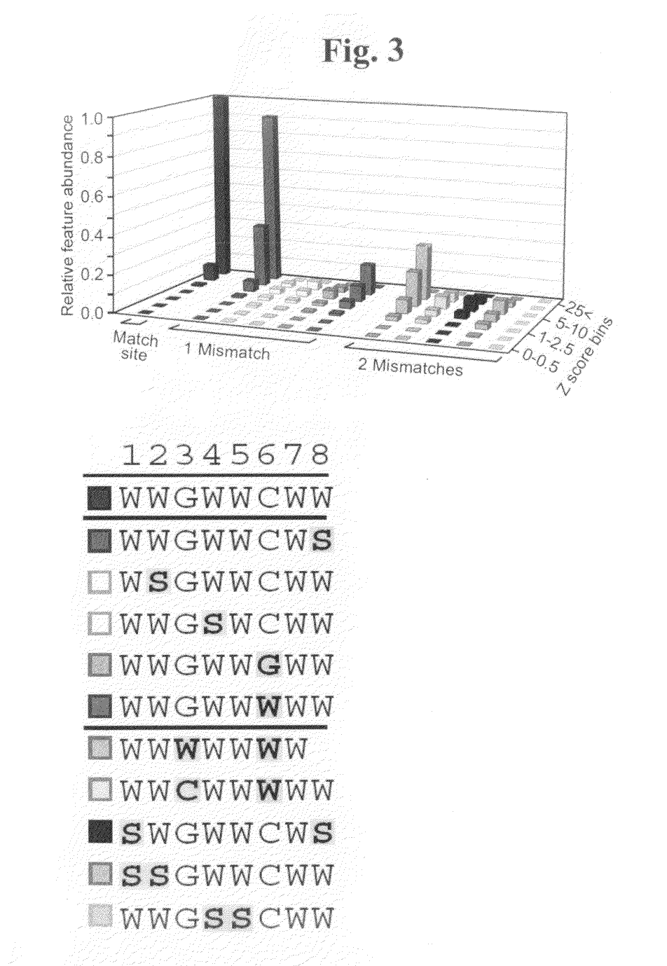

Comprehensive mutational analysis. In essence, the array performs a comprehensive “mutational” analysis as it queries the entire sequence space (within a defined size) to determine the contribution of every base pair in the cognate site for molecular recognition (FIG. 3).

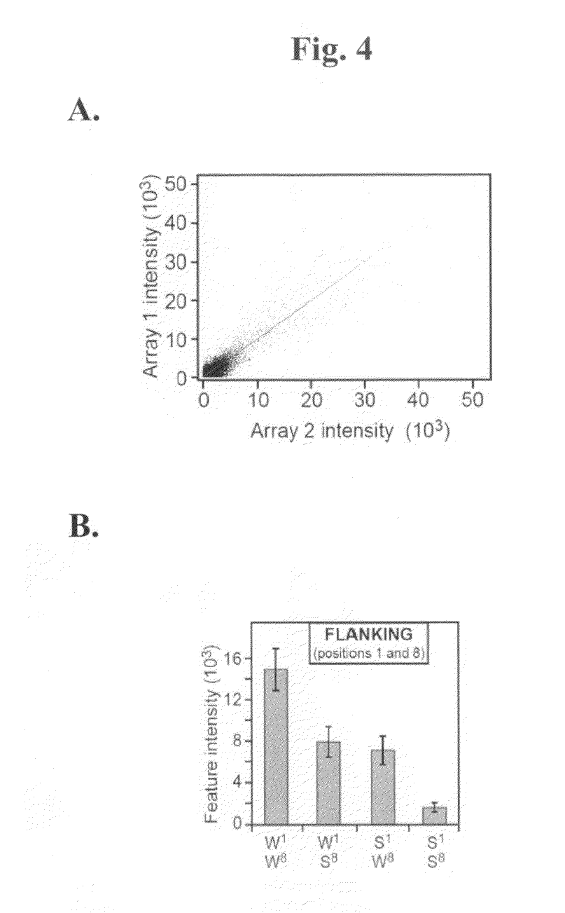

PA1 was incubated with the CSI array and a distinct pattern of fluorescent binding features was readily discernible, which did not change over a broad range of PA1 concentrations (0.5-500 nM). The array-to-array variability was very low with an average correlation coefficient of 0.88 (FIG. 4A). A majority of the features showed low background fluorescence and a small subset of the features were of high intensity (FIG. 2A, FIG. 3A). The duplicate features within an array and replicate features between arrays were averaged together to give finalized intensities. These averaged intensities were then converted into Z-scores [z=signal−meant/standard deviation] to reflect the signal-to-noise ratio.

By examining the array data, it is apparent that substituting an S (G or C) at position 8 only subtly decreases binding by PA1. This is consistent with the ability of this symmetric polyamide to bind the sequence in only one orientation. Replacing one of the S residues at positions 6 (or 3′) with a W significantly attenuates, but does not abolish binding. However, substituting any other position that prefers a W in the motif with an S residue nearly abolishes binding by PA1 (FIG. 3). The data also shows that despite a double substitution at positions 3 and 6 to W, the resulting A/T stretch retains its ability to bind PA1. This is likely a result of the inherent affinity of polyamides for A/T rich sequences (Kielkopf, C. L., White, S., Szewczyk, J. W., Turner, J. M., Baird, E. E., Dervan, P. B., Rees, D. C. (1998) Science 282, 111-115).

Data Processing: For each replicate, global mean normalization was used to ensure the mean intensity of each microarray was the same. Local mean normalization (Colantuoni, C., Henry, G., Zeger, S. & Pevsner, J. (2002) Bioinformatics 18, 1540-1541) was then used to ensure the intensity was evenly distributed throughout each sector of the microarray surface. Outliers between replicate features were detected using the Q-test at 90% confidence and filtered out. The replicates were then quantile normalized (Bolstad, B. M., Irizarry, R. A., Astrand, M. & Speed, T. P. (2003) Bioinformatics 19, 185-193) in order to account for any possible non-linearity between arrays. Duplicate features were then averaged together. The median of the averaged features was subtracted to account for background.

Z-scores were calculated as |signal−median|/standard deviation. Due to the right-handed tail effect, standard deviation of the background signal was based upon the standard deviation from the median of all signals less than the median. The relationship of Z-score to P-value can be found in Table 1, below. Motifs were then found by running several motif finding algorithms (Bailey, T. L. & Elkan, C. (1994) Proc. of the 2nd Intl. Conference on Intelligent Systems for Mol. Biol., AAAI Press, Menlo Park, Calif., 28-36; Hughes, J. D., Estep, P. W., Tavazoie, S. & Church, G. M. (2000) J. Mol. Biol. 296, 1205-1214; Liu, X. S., Brutlag, D. L. & Liu, J. S. (2002) Nat. Biotechnol. 20, 835-839) on sequences in the highest Z-score bin. Logos (Schneider, T. D. & Stephens, R. M. (1990) Nucl. Acids Res. 18, 6097-6100) of each motif were then created by using sequences from the highest Z-score bin that contained the motif.

Table 1, below, shows the P-value associated with each Z-score for assay molecule PA1 binding to the target array. The P-value represents the likelihood that the feature's true mean is zero. That is, the probability that the feature with that Z-score is not preferentially bound by the fluorescently-labeled ligand. Thus, for all features with a Z-score of 4.0, there is a false positive rate of 0.0064% (ie, 1 feature in 15625 is not preferentially bound by the ligand).

TABLE 1
P-value associated with Z-score for PA1

| Z-score | Probability | Likelihood |
|---|---|---|
| 25 | 6.12 × 10⁻¹³⁶ | 1.64 × 10¹³⁷ |
| 10 | 1.54 × 10⁻²³ | 6.55 × 10²³ |
| 5 | 5.74 × 10⁻⁷ | 1.74 × 10⁶ |
| 2.5 | 0.01242 | 80.5 |
| 1 | 0.31730 | 3.15 |
| 0.5 | 0.61708 | 1.62 |
| 0 | 1 | 1 |

Sequences in the highest Z-score bin (≧25) were subjected to several motif searching algorithms (Bailey, T. L. & Elkan, C. (1994) Proc. of the 2nd Intl. Conference on Intelligent Systems for Mol. Biol., AAAI Press, Menlo Park, Calif., 28-36; Hughes, J. D., Estep, P. W., Tavazoie, S. & Church, G. M. (2000) J. Mol. Biol. 296, 1205-1214; Liu, X. S., Brutlag, D. L. & Liu, J. S. (2002) Nat. Biotechnol. 20, 835-839) which identified 5′-W¹W²G³T⁴W⁵C⁶W⁷W⁸-3′, a motif that is nearly identical to the predicted binding site for the polyamide, 5′-WWGWWCWN-3′. Parsing of the core sequences (N²⁻⁷) showed that not all permutations of the consensus are bound equally well. In particular, all sequences that contained the sequence 5′-WWGATCWW-3′ had significantly lower intensities than other permutations of the consensus sequence. This observation is consistent with previous solution studies where this preference was first identified (White, S., Baird, E. E. & Dervan, P. B. (1996) Biochemistry 35, 12532-12537). Furthermore, the flanking sequence (N¹, N⁸) showed an equally strong preference for a W (A/T) in both positions (FIG. 4B). This is also in agreement with the preference of the polyamide γ-butyric acid (GABA) turn and dimethylaminopripylamide tail (Dp) for A/T residues (Swalley, S. E., Baird, E. E. & Dervan, P. B. (1999) J. Am. Chem. Soc. 121, 1113-1120).

The analysis of the CSI array binding data also showed that the cognate site preferences identified by the array were entirely consistent with reported solution binding studies of this polyamide for five different polyamide sequences (FIG. 2B and Table 2). The high correlation (r²=0.997) of feature intensity on the array with affinity for different cognate sites in solution provides significant confidence in the veracity of the cognate site preferences identified by the array. FIG. 2B compares the data from the CSI array binding (x-axis) to the solution data, shown in Table 2, below. Taken together, these correlations demonstrate that the CSI array correctly identifies the cognate sites of a DNA binding molecule, and it accurately ranks each cognate site in the order of increasing affinity.

Table 2, below, shows the data and references for FIG. 2B. The association constants, Kₐ, (equilibrium anity constants) were determined by footprinting. Intensities of the array binding data from this Example were determined by averaging together all features that contain the sequence used in the footprints. Data Point labels refer to FIG. 2B.

TABLE 2
Data for Kₐ versus intensity of each data point in FIG. 2B.

| Data Point | Sequence | Kₐ (10⁹ M⁻¹) | Intensity | Reference |
|---|---|---|---|---|
| A | ATGTACAT | 70 | 19715 | Foister, S, et al. Bioorg. Med. |
| | | | | Chem., 11, 4333-4340 (2003) |
| B | AGTACT | 37 | 10952 | Trauger, J W, et al., Nature, |
| | | | | 382, 559-561 (1996) |
| C | ATCTACAT | 3.2 | 3035.5 | Foister, S, et al., Bioorg. |
| | | | | Med. Chem., 11, 4333-4340 (2003) |
| D | ATATACAT | 2.8 | 2073.25 | Foister, S, et al., Bioorg. Med. |
| | | | | Chem., 11, 4333-4340 (2003) |
| E | ATGTATTT | 0.41 | 1426.5 | Baird, E E, Dervan, P B., |
| | | | | California Institute of |
| | | | | Technology, Ph.D. Thesis, 1999 |

### Example 3

### Array Binding of a Transcription Factor and a Cooperative Assembly

Example 2 demonstrates the accuracy of the CSI arrays in the assay of binding of a nucleic acid binding assay molecule to a nucleic acid target array. This example, Example 3, demonstrates the assay of sequence preferences for assay molecules that bind DNA cooperatively. In this example, the cognate site preference of Extradenticle (Exd) is assayed. Exd is, a transcription factor that plays an essential role in Drosophila development and is highly conserved across species, including humans (Rauskolb, C., Peifer, M. & Wieschaus, E. (1993) Cell 74, 1101-1112). Exd binds DNA cooperatively with Hox-family transcription factors (Rauskolb, C., Peifer, M. & Wieschaus, E. (1993) Cell 74, 1101-1112; Mann, R. S. & Chan, S. K. (1996) Trends Genet. 12, 258-262). This interaction increases the sequence specificity and affinity of Hox proteins for DNA. Structural studies of the Hox-Exd-DNA ternary complexes show a Hox peptide (YPWM) docked on the DNA binding domain of Exd. (Passer, J A M, et al., Nature 1999, 397:714-9)

Individual Hox-proteins as well as Exd bind DNA with very low affinity and with poor specificity (Mann, R. S. & Chan, S. K. (1996) Trends Genet. 12, 258-262). Cooperative binding dramatically increases the affinity of Exd and Hox proteins for DNA and strongly influences DNA sequence specificity such that one Hox-Exd complex targets different genes than another Hox-Exd complex (Mann, R. S. & Chan, S. K. (1996) Trends Genet. 12, 258-26246).

For this example, synthetic molecules (polyamide-peptide conjugates) were generated that can mimic two key functions of the Hox-family of transcription factors (Arndt, H., Hauschild, K., Sullivan, D., Lake, K., Dervan, P., Ansari, A. (2003) J. Am. Chem. Soc. 125, 13322-13323). First, they can bind sites targeted by specific Hox proteins, and second, they can cooperatively recruit Exd to an adjacent cognate site.

Molecular Modeling: Molecular models were created by aligning the DNA from Protein Database (pdb) files of Exd crystallized with DNA (1B8I) to hairpin polyamide crystallized with DNA (1M18) (Berman, H. M., Westbrook, J., Feng, Z., Gilliland, G., Bhat, T. N., Weissig, H., Shindyalov, I. N., Bourne, P. E. (2000) Nucl. Acids Res. 28, 235-242). The DNA was aligned at four different positions using structural alignment software (Guex, N. & Peitsch, M. C. (1997) Electrophoresis 18, 2714-2723) so as to create Consensus, Consensus+1, Consensus−1, and Inverse binding of the polyamide relative to Exd. The distance from the N-methyl group of the heterocycle ring that is analogous to the N-methyl group of the ring of our polyamide which bears the hexapeptide to the carboxylcarbon of the methionine of the recruitment peptide bound to Exd in the crystal structure was then calculated for each of the four alignments. This demonstrated the distance the linker in our polyamide (PA2 and PA3) would have to reach in order to recruit Exd to DNA (Guex, N. & Peitsch, M. C. (1997) Electrophoresis 18, 2714-2723). The alignments were visualized using Visual Molecular Dynamics Software (VMD) (Humphrey, W., Dalke, A. & Schulten, K. (1996) J. Mol. Graphics 14, 33-38). The linkers for PA2 and PA3 were then drawn and energy minimized to estimate how far each linker could likely reach (ChemDraw Ultra 9.0. (2005) CambridgeSoft).

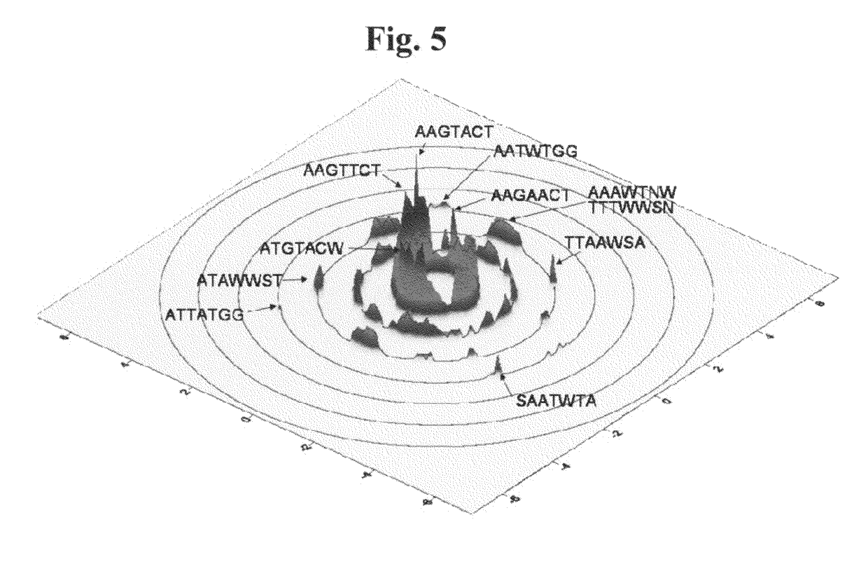

A synthetic Hox mimic was constructed that is a polyamide-FYPWM conjugate (PA-YPWM; using the standard single-letter amino acid code to represent the amino acid residues). PA-YPWM was found to mimic two key Hox functions: (i) it binds to a site adjacent to the Exd binding site and (ii) it recruits Exd to its cognate site as efficiently as the natural Hox partner. Gel mobility shift assays of PA-YPWM mimics (shown in FIG. 5) with variations in the linker between them showed that linkers longer than 7 Angstroms function as well as the optimal linker conjugate 3. It is believed that the flexible linker permits attaching of “docking” peptides or small molecules that interact with unmapped surfaces of other TFs.

Electrophoretic Mobility Shift Assays: 40mer DNA sequences labeled with ³²P (as per standard methods) were used in all reactions. Reactions were performed in a buffer composed of 150 mM Potassium Glutamate, 50 mM HEPES pH=7.5, 2 mM DTT, 100 ng/μL of BSA, 10% DMSO, and 10% glycerol. For the binding tests, polyamide-peptide conjugates (50 nM final concentration) and ³²P-DNA were incubated together for 30 minutes at 4° C. Exd was then added to bring the reaction volume to 20 μL. Exd final concentrations were 0.033, 0.1, 0.33, 1, 3.3, 10, 33, and 100 nM. These reactions were incubated at 4° C. for 1 hour and then 15 μL was loaded onto a pre-run 10% acrylamide/3% glycerol gel (1×TBE).

Hairpin Induction for Electrophoretic Mobility Shift Assay (Supplementary Figure S4B). Hairpin DNA was formed by heating synthesized ssDNA to 95° C. for 5 minutes and then rapidly cooling them on ice. The 34mer DNA hairpin polyamides were then labeled with ³²P (as per standard methods). For the hairpin electrophoretic mobility shift assays the concentrations of polyamide used were 50 nM, and Exd was present at a concentration of 100 nM. The reactions were performed in the above buffer and the gels were then run as before with the following exceptions: the gels were 8% acrylamide/3% glycerol gel with 0.5×TBE.

### Consensus DNA:

5′-CGC TGATTGAC CGC TCCT GCG GTCAATCA GCG TTTT-3′

### G-Rich DNA:

5′-CGC CCCCCCCC CGC TCCT GCG GGGGGGGG GCG TTTT-3′

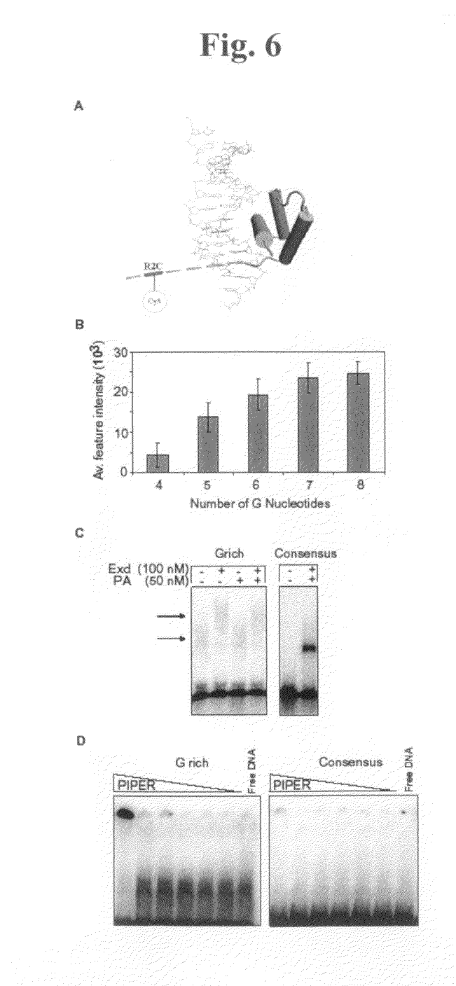

Piper Electrophoretic Mobility Shift Assay (FIG. 6D). The G-rich and Consensus hairpin DNA (same DNA as used in Hairpin Induction for EMSA) were incubated with increasing PIPER (Humphrey, W., Dalke, A. & Schulten, K. (1996) J. Mol. Graphics 14, 33-38) concentrations. PIPER final concentrations were 0.15, 0.30, 0.625, 1.25, 2.5, and 5 mM. The gels were run and processed as above using an 8% acrylamide/3% glycerol gel with 0.5×TBE.

Gel Mobility Shift Results: Gel mobility shift assays with variation in temperature showed that the transcriptional activation with a Hox mimic was a temperature sensitive chemical switch. This strategy can be used to confer temperature sensitivity on other composite molecules. Ligand-responsive aptamer linkers can be used to connect fragments of a molecule and externally control its function.

To determine the sequence specificity of Exd, we labeled it with Cy3 at a unique cysteine residue on an unstructured portion of the protein (FIG. 6) (Passner, J. M., Ryoo, H. D., Shen, L., Mann, R. S. & Aggarwal, A. K. (1999) Nature 397, 714-719). The modified protein does not differ in its ability to bind cooperatively with a member of the Hox family (Ubx) or with a synthetic Hox mimic to a known cognate DNA site.

Dye conjugation to Exd: A pET3A vector containing the Exd sequence [residues 1-88] was mutated using standard quick change mutagenesis procedures to replace the cysteine with a serine (C41S) and an arginine was replaced with a cysteine (R2C) to generate Exd R2C (see FIG. 6A). This Exd mutant was found to be stable and the mutation had a minimal effect on DNA binding affinity (M. Brezinski, unpublished data). Exd R2C was then labeled with Cy3 using Amersham Biosciences Cy3 Maleimide Mono-Reactive Dye Pack (P#: PA23031). The molar Dye/Protein ratio was determined to be 0.96 (quantified as shown below).

[Cy3]=(A₅₅₂×Dilution factor)/150000 M⁻¹cm⁻¹

[Exd R2C]=[A₂₈₀−(0.08×A₅₅₂)]/12090 M⁻¹cm⁻¹

D/P=[Cy3]/[Exd R2C]

Exd sequence: ¹A(R->C)RKRRNFSK ¹¹QASEILNEYF ²¹YSHLSNPYPS ³¹EEAKEELARK ⁴¹(C->S)GITVSQVSN ⁵¹WFGNKRIRYK ⁶¹KNI

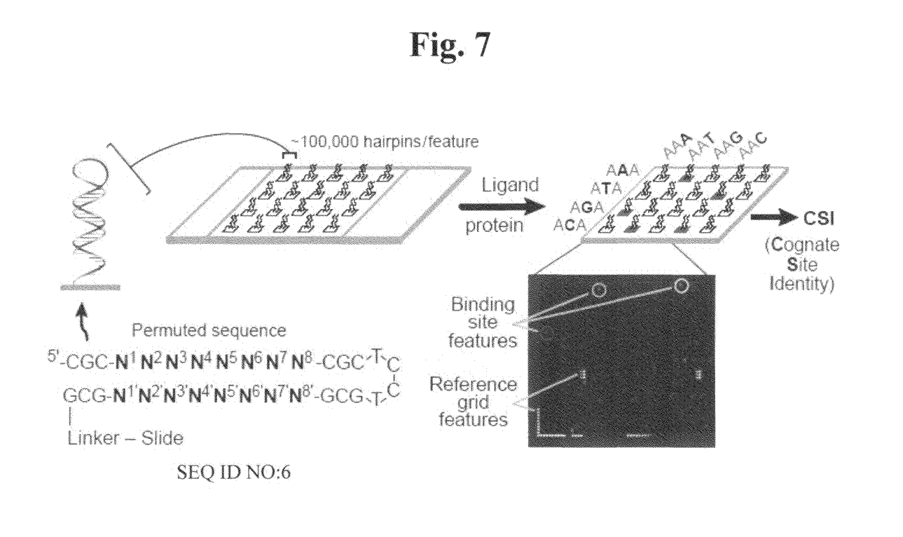

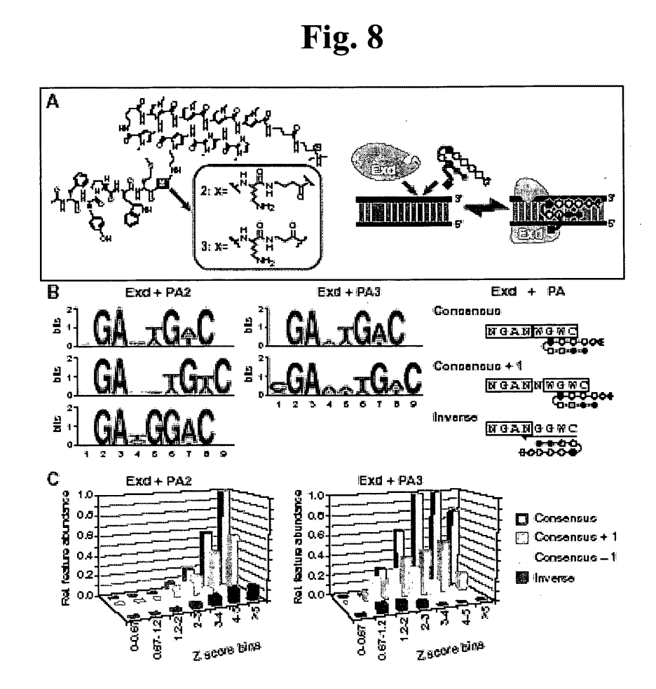

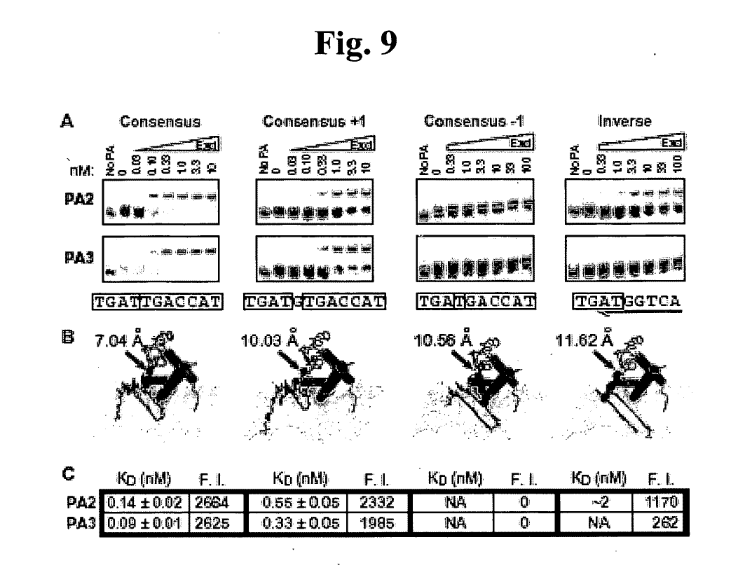

Array design for Hox/Exd Cooperative Binding Assay. The duplex DNA sequences are designed as self-complementary palindromes interrupted at the center by a TCCT sequence to facilitate the formation of DNA hairpins (FIG. 7). The 34 residue oligonucleotide is synthesized directly on the glass surface using a maskless array synthesizer (Singh-Gasson, S., Green, R. D., Yeu, Y., Nelson, C., Blattner, F., Sussman, M. R., Cerrina, F. (1999) Nat. Biotechnol. 17, 974-978) that can readily create up to 786,000 spatially resolved features. The unimolecular construction of duplex DNA allows the array to be reused several times without appreciable loss of information. After inducing hairpin formation, we find that greater than 95% of the oligonucleotides in the array form duplexes. In our hairpin design, we added three constant base pairs on either side of the 8 base pairs that were permuted (N′-8). Previous work shows that this is sufficient to buffer the core of the hairpin stem against thermal end fraying of the duplex and against deviations from B-form DNA due to the presence of the loop (Ansari, A., Kuznetsov, S. V. & Shen, Y. (2001) Proc. Natl. Acad. Sci. U.S.A. 98, 7771-7776). Several lines of evidence have demonstrated that the core of a hairpin stem interacts with proteins and nucleic acid binding small molecules indistinguishably from DNA duplexes composed of two individual complementary strands (Kim, Y., Geiger, J. H., Hahn, S. & Sigler, P. B. (1993) Nature 365, 512-520; Tse, W. C., Ishii, T. & Boger, D. L. (2003) Bioorg. Med. Chem. 11, 4479-4486).

P-value associated with each Z-score for Exd binding cooperatively to the target array is shown in Table 3, below. The P-value represents the likelihood that the feature's true mean is zero. That is, the probability that the feature with that Z-score is not preferentially bound by the fluorescently-labeled ligand. Thus, for all features with a Z-score of 4.0, there is a false positive rate of 0.0064% (ie, 1 feature in 15625 is not preferentially bound by the ligand).

TABLE 3
P-value associated with Z-score for Exd binding

| Z-score | Probability | Likelihood |
|---|---|---|
| 5 | 5.74 × 10⁻⁷ | 1.74 × 10⁶ |
| 4 | 6.40 × 10⁻⁵ | 15625 |
| 3 | 0.002698 | 371 |
| 2 | 0.0455 | 21.9 |
| 1.2 | 0.23014 | 4.35 |
| 0.67 | 0.50285 | 1.99 |
| 0 | 1 | 1 |

FIG. 8 shows data from the assay of a cooperative assembly of assay molecules to a DNA hairpin target array as discussed in Example 3. CSI-arrays were used to determine the DNA binding preference for the Hox mimic, as shown in FIG. 8C, with the consensus in FIG. 8B. The synthetic mimic, Hox-5, was selected from these studies to be injected into Drosophila, and was found to have biological activity mimicking Hox in vivo. This was an example of the use of the methods and systems of the present invention to identify and select an artificial nucleic acid binding molecule that had the desired biological activity.

When tested on the CSI array, Exd alone, as expected, demonstrated little sequence-specific binding at concentrations ranging from 0.2-200 nM. It does, however, show an unexpected preference for stretches of consecutive G-residues. Initial studies suggest that these sequences can form non-B-form, likely G-quadruplex (Sen, D., & Gilbert, W. (1992) Methods Enzymol., 211, 191-199) structures. As a result of CSI array analysis, the physiological importance of this binding interaction was identified for future study.

When incubated with two different synthetic Hox mimics (PA2 and PA3), the Cy3-labeled Exd displayed an unambiguous pattern of feature binding in both sets of experiments. PA2 and PA3 (ImImPy*Py-γ-ImPyPyPy-β-Dp) are designed to target the sequence 5′-WGWCCW-3′. Furthermore, instead of a Cy3 dye conjugated to the polyamide as in PA1, PA2 and PA3 do not bear any dye but are conjugated to an Exd binding peptide (N-FYPWMK-C). PA2 and PA3 differ solely by a single methylene in the linker connecting the Exd binding peptide to the polyamide (FIG. 8A) (Hauschild, K. E., Metzler, R. E., Arndt, H. D., Moretti, R., Raffaelle, M., Dervan, P. B., Ansari, A. Z. (2005) Proc. Natl. Acad. Sci. U.S.A. 102, 5008-5013). Since these polyamides are not fluorescently labeled, we detect cognate sites bound cooperatively by synthetic Hox mimics and Exd, as well as sites bound by Exd alone.

The raw array data for the above experiments were treated as described in Example 2. In addition to the G-stretches that Exd binds in the absence of any partner, three clear motifs emerged from the PA2-Exd data, whereas only two of those motifs were found in the PA3-Exd data (FIG. 8B). The Exd binding motif is 5′-NGAN-3′, which is consistent with the structural and genetic studies of Hox-Exd cognate sites (White, R. A., Aspland, S. E., Brookman, J. J., Clayton, L. & Sproat, G. (2000) Mech. Dev. 91, 217-226). In other words, the 5′-GA-3′ dinucleotide is the only required sequence determinant for Exd binding to DNA. Remarkably, the array identified the differences in the arrangement of polyamide and Exd binding sites due to ˜1.25 Å difference in the linker length between PA2 and PA3 (FIG. 8C). The other important result that emerges is that cooperative ternary assembly with Exd stabilizes binding of synthetic Hox mimics to truncated sites (5′-WGWC-3′). This is often seen in nature, where cooperative assembly of transcription factors utilizes sub-optimal binding sites to ensure that only a higher order complex can efficiently bind to a regulatory element (Thanos, D. & Maniatis, T. (1996) Methods Enzymol. 274, 162-173; Ptashne, M. & Gann, A. Genes and Signals. (2002) CSHL Press, New York).

Solution binding and molecular modeling. To validate the unexpected differences in the motifs identified by each polyamide with Exd, we performed electrophoretic mobility shift assays (EMSA). These studies with Exd and the two Hox-mimics strongly support the cognate site preferences identified by the array (FIG. 8A and FIG. 8C). Furthermore, molecular modeling (Guex, N. & Peitsch, M. C. (1997) Electrophoresis 18, 2714-2723; Humphrey, W., Dalke, A. & Schulten, K. (1996) J. Mol. Graphics. 14, 33-38) analyses of PA2 and PA3 with Exd (with a docked Hox hexapeptide) agree well with the CSI array data. Both demonstrate that the linkers for PA2 and PA3 (9.98 and 11.25 Å, respectively) are able to deliver the hexapeptide to Exd at the composite consensus site (FIG. 9B). The fully extended linkers reach Exd at the gapped composite site (consensus+1); however, simple geometric measurements with some energy minimization (ChemDraw Ultra 9.0. (2005) CambridgeSoft) suggest that the linker of PA3 should not reach. In the case of inverted binding sites, it is clear from modeling that the linker of PA3 is incapable of reaching Exd, and that the linker of PA2, even when fully extended, would be suboptimal yielding an unstable ternary complex with Exd. These predictions are in good agreement with the observed CSI array binding data and electrophoretic mobility shift assay results (FIG. 9A and FIG. 9C). However, the array data also demonstrate that a single base overlap (consensus−1) in the binding sites is not able to support binding of the complex, despite the fact that modeling indicates the distance is similar to that of the consensus+1 site (FIG. 9B). The binding of either partner to overlapping sites may deform the DNA and prevent complex formation even though modeling studies suggest that polyamide or Exd binding to the consensus−1 site should not disfavor complex formation (Passner, J. M., Ryoo, H. D., Shen, L., Mann, R. S. & Aggarwal, A. K. (1999) Nature 397, 714-719; LaRonde-LeBlanc, N. A. & Wolberger, C. (2003) Genes Dev. 17, 2060-2072). The ambiguities in molecular docking and energy minimization methods thus prevent precise prediction of geometry of DNA grooves and distances between the interacting partners. In other words, the dramatic consequences on cognate site preference due to subtle, seemingly trivial, alterations in the linker length would not be readily apparent without the CSI array analysis. Therefore, this approach provides unexpected insight into molecular recognition properties of DNA binding molecules when they bind individually or in cooperative pairs.

We claim:

1. A target molecule that comprises:
L¹-X¹-L²-X²
where
L¹ is a polynucleotide from 8 to 30 nucleotides in length;
X¹ is a polynucleotide from 4 to 100 nucleotides in length, wherein from 1 to 3 of the first three nucleotide residues in the sequence of X¹ and 1 to 3 of the last three nucleotide residues in the sequence of X¹ are G or C;
L² is a polynucleotide three nucleotides in length, having the sequence GNA, where N is A, G, C, T, or U;
X² is a polynucleotide from 4 to 100 nucleotides in length, wherein the residues from the 3′ region to the 5′ terminus of X² are 75% to 100% complementary to the corresponding residues in X¹, and the portion of X² that that are 90-100% complementary to X¹.

2. The target molecule according to claim 1 wherein L¹ is from 10 to 20 nucleotides in length.

3. The target molecule according to claim 2 wherein L¹ is 15 nucleotides in length.

4. The target molecule according to claim 1 wherein X¹ is from 6 to 30 nucleotides in length.

5. The target molecule according to claim 4 wherein X¹ is from 15 to 26 nucleotides in length.

6. The target molecule according to claim 1 wherein X¹ and X² comprise deoxyribonucleotide residues.

7. The target molecule according to claim 1 wherein X¹ and X² comprise ribonucleotide residues.

8. An array comprising target molecules according to claim 1.

9. The array according to claim 8 comprising from 1 to 2 million target molecules.

10. The array according to claim 8 wherein the combined target molecules represent all permutations of a 10-nucleotide long nucleic acid sequence.

11. The array according to claim 10 wherein the combined target molecules represent all permutations of an 8-nucleotide long nucleic acid sequence.

12. A method of selecting nucleic acid-binding molecules comprising the steps of:
a. providing a target array that comprises a solid surface attached to an array of target molecules according to claim 1;
b. reducing non-specific binding by pre-treating the target array with a non-specific blocker selected from the group consisting of silanizing agents, alkylating agents, protein, and nucleic acid;
c. providing an assay solution that comprises potential nucleic acid binding molecules;
d. contacting the target array with the assay solution under conditions to permit binding of a target molecule with a potential nucleic acid binding molecules; and
e. determining the binding of nucleic acid binding molecules to the target molecules in the target array.


SEQUENCE LISTING (6 sequences)

SEQ ID NO: 1 - protein, 6 aa, Drosophila melanogaster
FYPWMK

SEQ ID NO: 2 - DNA, 32 bp, Artificial Sequence
  synthetic hairpin-forming DNA sequence
cgcttagttcacgctcctgcgtgaactaagcg

SEQ ID NO: 3 - DNA, 32 bp, Artificial Sequence; identical to SEQ ID NO: 2
  synthetic hairpin-forming DNA sequence

SEQ ID NO: 4 - DNA, 36 bp, Artificial Sequence
  synthetic hairpin-forming DNA sequence
cgccccccccccgctcctgcggggggggggcgtttt

SEQ ID NO: 5 - protein, 63 aa, Drosophila melanogaster
ARRKRRNFSKQASEILNEYFYSHLSNPYPSEEAKEELARKCGITVSQVSNWFGNKRIRYKKNI

SEQ ID NO: 6 - DNA, 32 bp, Artificial Sequence
  synthetic hairpin-forming DNA sequence
cgcnnnnnnnncgctcctgcgnnnnnnnngcg